(19) United States
(12) Patent Application Publication
(10) Pub. No.: US 2019/0321477 A1
(43) Pub. Date: Oct. 24, 2019

(54) METHODS AND COMPOSITIONS RELATED TO CHITOSAN-DERIVED NANOPARTICLE-MEDIATED CRISPR/CAS9 DELIVERY

(71) Applicant: BOARD OF REGENTS, THE UNIVERSITY OF TEXAS SYSTEM, Austin, TX (US)

(72) Inventors: Hugh D.C. Smyth, Austin, TX (US); Hairui Zhang, Austin, TX (US); Silvia Ferrati, Austin, TX (US)

(21) Appl. No.: 16/313,255

(22) PCT Filed: Jun. 27, 2017
(86) PCT No.: PCT/US2017/039602

§ 371 Date: Dec. 26, 2018

Related U.S. Application Data
(60) Provisional application No. 62/355,037, filed on Jun. 27, 2016.

(51) Int. Cl. A61K 47/36; A61K 47/10; A61K 9/51; C12N 15/113

(52) U.S. Cl. CPC A61K 47/36; A61K 47/10; A61K 9/5161; C12N 2800/80; C12N 2310/14; C12N 2310/20; C12N 15/113

(57) ABSTRACT

Disclosed herein are nucleic acid delivery systems and methods of use.

### FIELD OF THE INVENTION

The present invention generally relates to systems, methods and compositions for use in PEG modified chitosan (PEG-g-Cs) and oligochitosan (PEG-g-OCs) nanoparticle-mediated delivery of CRISPR/Cas9 systems.

### BACKGROUND

Cystic fibrosis (CF) is the most common lethal genetic disease in the white population caused by mutations in the cystic fibrosis transmembrane conductance regulator (CFTR) which regulates chloride transport across epithelia barriers. The defect of CFTR protein lead to reduced secretion of both chloride and bicarbonate, which impedes mucosal clearance. Consequently, there would be chronic airway and systemic inflammation, tissue destruction, and respiratory insufficiency. Approximately 1900 disease-causing CFTR mutations have been found, but only a small number of them have been studied well. The most common mutation, accounting for about two-thirds of mutated alleles in northern European and North American populations, is a deletion of phenylalanine at position 508 (F508del) in exon 11 on chromosome 7.

Although CF is one of the most well studied genetic diseases, current treatment of patients with CF focuses on managing symptoms rather than correcting the genetic mutation. Some studies have focused on treatments which can alleviate the symptoms in CF patients. It was found that ivacaftor (also known as VX-770 and Kalydeco) increased chloride transport in patients having the G551D variant up to 50% of wild-type level and oflumacaftor (also known as VX-809) restored activity of CFTR to approximately 14% of non-CF human bronchial epithelial cells. Histone deacetylase inhibitors enhanced function of chloride channel to 28% of those with wild-type CFTR.

Gene therapy is probably the only way to cure CF fundamentally. However, because of the difficulty of delivery to lung and safety issues, gene therapy is still unsuccessful. What are needed are new systems, methods, and compositions that can safely deliver corrective gene therapy to the lungs of CF patients.

### SUMMARY

Disclosed herein is a nucleic acid delivery system comprising: a CRISPR-Cas9 polynucleotide sequence, wherein the polynucleotide sequence comprises a guide sequence capable of hybridizing to a target sequence in a eukaryotic cell; donor-corrected nucleic acid; and a polymeric carrier system, wherein the polymeric carrier comprises polyethylene glycol (PEG) bound to chitosan, further comprising an oligo-chitosan backbone. Also disclosed are methods of transforming cells and methods of treatment using the system disclosed herein.

In one aspect disclosed herein are polymer carriers comprising polyethylene glycol (PEG) chemically bound to a chitosan or oligo-chitosan backbone.

Also disclosed herein are nucleic acid delivery system comprising: a CRISPR-Cas9 polynucleotide sequence, wherein the polynucleotide sequence comprises a guide sequence capable of hybridizing to a target sequence in a eukaryotic cell; donor-corrected nucleic acid; and a polymeric carrier system, wherein the polymeric carrier comprises polyethylene glycol (PEG) bound to a chitosan or oligo-chitosan backbone.

In one aspect are nucleic acid delivery systems, polymer carriers, and polymer carrier systems of any preceding aspect, wherein the delivery system forms nanoparticles.

In one aspect are nucleic acid delivery systems, polymer carriers, and polymer carrier systems of any preceding aspect, wherein the donor-corrected nucleic acid is siRNA.

In one aspect are nucleic acid delivery systems, polymer carriers, and polymer carrier systems of any preceding aspect, wherein the donor-corrected nucleic acid corrects an F508del CFTR mutation.

In one aspect are nucleic acid delivery systems, polymer carriers, and polymer carrier systems of any preceding aspect, wherein the delivery particles are less than 500 nm in diameter, less than 250 nm in diameter, or less than 100 nm in diameter.

Also disclosed are nucleic acid delivery systems, polymer carriers, and polymer carrier systems of any preceding aspect, wherein the nanoparticle formulation comprises a lipid-based nanoparticle.

In one aspect, disclosed herein are nucleic acid delivery systems, polymer carriers, and polymer carrier systems of any preceding aspect, wherein the ratio of moles of the amine groups of chitosan to that of the phosphate of DNA (N/P ratio) is less than 1.

Also disclosed are method of modifying an organism or a non-human organism by manipulation of a target sequence in a genomic locus of interest comprising administering a composition comprising the nucleic acid deliver systems, polymer carriers, and polymer carrier systems of any preceding aspect.

In one aspect, disclosed herein are methods of treating a subject with cystic fibrosis, the method comprising administering the nucleic acid deliver systems, polymer carriers, and polymer carrier systems of any preceding aspect.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows an illustration of complex formation using the systems PEG-g-OCs and PEG-g-Cs and siRNA.

FIGS. 2A, 2B, 2C, 2D, and 2E show the FT-IR spectra of (a) chitosan, (b) m-PEG, (c) NPHOCs, (d) PEG-g-NPHOCs and (e) PEG-g-OCs.

FIGS. 3A, 3B, 3C, 3D, and 3E show the FT-IR spectra of (a) chitosan, (b) m-PEG, (c) NPHCs, (d) PEG-g-NPHCs and (e) PEG-g-Cs.

FIG. 4 shows the FT-IR spectra of a) oligo chitosan, b) mPEG-5000, c) mPEG-COOH, d) NPHOC, e) PEG-g-NPHOC, f) PEG-g-OC (DS=5%) and g) PEG-g-OC (DS=50%).

FIGS. 5A, 5B, 5C, 5D, 5E, and 5F show H1 NMR spectra of oligo chitosan, mPEG-COOH, NPHOC, PEG-NPHOC in DMSO, mPEG-g-OC (DS=5%) and mPEG-g-OC (DS=50%) in D₂O (1% DCl).

FIGS. 6A and 6B show the binding efficiency between pegylated chitosan and DNA at different N/P ratios. Lane 1: Ladder, Lane 2: naked DNA, Lanes 3-9: N/P=0.5, 1, 2, 5, 10, 20 and 30 respectively. a) PEG-g-OC and b) PEG-g-Cs.

FIG. 7A shows a displacement assay showing that DNA successfully released from the complexes.

FIG. 7B shows the protection capacity against DNase I enzymatic digestion

FIG. 8 shows the effect of N/P ratio on the size of PEG-g-Cs/DNA complexes.

FIG. 9 shows the effect of N/P ratio on the zeta potential of PEG-g-Cs/DNA complexes.

FIG. 10 shows gel electrophoresis analysis of nanocomplex's mucus-penetration ability. Lane 1: ladder, lane 2: negative control (mucus only), lane 3: naked DNA, lane 4: N/P ration=0.5, lane 5: N/P ratio=1, lane 6: N/P ratio=5.

FIG. 11 shows a transfection assay in media with different pH in HEK 293 cells (N/P ratio=20) (Control is last panel).

### DETAILED DESCRIPTION OF THE INVENTION

Disclosed herein are systems, methods, and compositions that relate to PEG modified chitosan (PEG-g-Cs) and oligochitosan (PEG-g-OCs) nanoparticle-mediated delivery of CRISPR/Cas9 systems to lung epithelia cells in order to correct the F508del CFTR mutation.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this invention belongs. The following definitions are provided for the full understanding of terms used in this specification.

### Terminology

As used herein, the article “a,” “an,” and “the” means “at least one,” unless the context in which the article is used clearly indicates otherwise.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers. In some embodiments, the polynucleotide is composed of nucleotide monomers of generally greater than 100 nucleotides in length and up to about 8,000 or more nucleotides in length.

The term “polypeptide” refers to a compound made up of a single chain of D- or L-amino acids or a mixture of D- and L-amino acids joined by peptide bonds.

The term “complementary” refers to the topological compatibility or matching together of interacting surfaces of a probe molecule and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The term “anneal” refers to the process by which a single-stranded nucleic acid sequence pairs by hydrogen bonds to a complementary sequence, forming a double-stranded nucleic acid sequence, including the reformation (renaturation) of complementary strands that were separated by heat (thermally denatured).

The term “melting” refers to the denaturation of a double-stranded nucleic acid sequence due to high temperatures, resulting in the separation of the double strand into two single strands by breaking the hydrogen bonds between the strands.

The term “target” refers to a molecule that has an affinity for a given probe. Targets can be naturally-occurring or man-made molecules. Also, they can be employed in their unaltered state or as aggregates with other species.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which can also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1a promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit 0-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) can include a promoter that is heterologous to the second nucleic acid (e.g. polynucleotide) as the result of human manipulation (e.g., by methods described in Sambrook et al., Molecular Cloning-A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette can comprise nucleic acids (e.g. polynucleotides) combined in such a way that the nucleic acids (e.g. polynucleotides) are extremely unlikely to be found in nature. For instance, human manipulated restriction sites or plasmid vector sequences can flank or separate the promoter from the second nucleic acid (e.g. polynucleotide). One of skill will recognize that nucleic acids (e.g. polynucleotides) can be manipulated in many ways and are not limited to the examples above.

The term “expression cassette” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) can include a promoter that is heterologous to the second nucleic acid (e.g. polynucleotide) as the result of human manipulation (e.g., by methods described in Sambrook et al., Molecular Cloning-A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) can include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The phrase “codon optimized” as it refers to genes or coding regions of nucleic acid molecules for the transformation of various hosts, refers to the alteration of codons in the gene or coding regions of polynucleic acid molecules to reflect the typical codon usage of a selected organism without altering the polypeptide encoded by the DNA. Such optimization includes replacing at least one, or more than one, or a significant number, of codons with one or more codons that are more frequently used in the genes of that selected organism.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

As used throughout, by a “subject” (or a “host”) is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human.

### DESCRIPTION

In one aspect, disclosed herein is a nucleic acid delivery system comprising: a CRISPR Cas9 polynucleotide sequence, wherein the polynucleotide sequence comprises a guide sequence capable of hybridizing to a target sequence in a eukaryotic cell; donor-corrected nucleic acid; and a polymeric carrier system, wherein the polymeric carrier comprises polyethylene glycol (PEG) bound to chitosan, further comprising an oligo-chitosan backbone. Also disclosed are methods of transforming cells, and methods of treatment using this system. This system can be used, for example, in lung epithelia cells in order to correct the F508del CFTR mutation.

Despite difficulties of delivering gene therapy to the lungs and safety concerns, gene efforts on employing gene therapy to treat Cystic fibrosis (CF) have not been abandoned.

In one aspect, disclosed herein is are polymer carriers and polymer carrier systems that utilize cationic polymer nanoparticles to deliver nucleic acid. This type of system can successfully deliver nucleic acid such as siRNA and plasmid, due to their positive surface charge and high loading capacity. Chitosan is a natural polymer that has shown to be biocompatible, biodegradable and non-toxic. In addition, when protonated under acidic conditions chitosan is able to form complexes with siRNA by electrostatic interactions. Due to these properties chitosan has been widely investigated as a potential carrier for gene delivery. By chemically binding chitosan or oligo-chitosan to polyethylene glycol (PEG) to form a PEG-chitosan or PEG-oligo-chitosan advantage can be taken of both Chitosan's ability to complex with nucleic acid and PEG's ability to accelerate transport of nanoparticles across epithelium. Accordingly, in one aspect, disclosed herein are polymer carriers comprising polyethylene glycol (PEG) chemically bound to a chitosan or oligo-chitosan backbone.

Different gene editing technologies including transcription activator-like effector nucleases (TALEN), zinc finger nucleases (ZFNs) and clustered regularly interspaced short palindromic repeats/associated (CRISPR/Cas) nucleases have been used to correct mutations in cystic fibrosis transmembrane conductance regulator (CFTR). In addition, McNeer et al. delivered triplex-forming peptide nucleic acid molecules to correct F508del-CFTR in cystic fibrosis bronchial epithelial (CFBE) cells.

It is understood and herein contemplated that the disclosed PEG-chitosan and/or PEG-oligo-chitosan carriers can be combined with gene editing technologies including transcription activator-like effector nucleases (TALEN), zinc finger nucleases (ZFNs) and clustered regularly interspaced short palindromic repeats/associated (CRISPR/Cas) nucleases to form nucleic acid delivery systems. In one aspect, disclosed herein are nucleic acid delivery systems comprising: a CRISPR-Cas9 polynucleotide sequence; donor-corrected nucleic acid; and a polymeric carrier system, wherein the polymeric carrier comprises polyethylene glycol (PEG) bound to a chitosan, or an oligo-chitosan backbone. Also disclosed are methods of transforming cells and methods of treatment using the system disclosed herein.

In one aspect, the nucleic acid delivery systems comprising a CRISPR-Cas9 polynucleotide sequence can be designed to target eukaryotic cells. For example, in one aspect, disclosed herein are nucleic acid delivery systems comprising a CRISPR-Cas9, wherein the polynucleotide sequence comprises a guide sequence capable of hybridizing to a target sequence in a eukaryotic cell; donor-corrected nucleic acid. Accordingly, disclosed herein are nucleic acid delivery systems comprising: a CRISPR-Cas9 polynucleotide sequence, wherein the polynucleotide sequence comprises a guide sequence capable of hybridizing to a target sequence in a eukaryotic cell; donor-corrected nucleic acid; and a polymeric carrier system, wherein the polymeric carrier comprises polyethylene glycol (PEG) bound to a chitosan or an oligo-chitosan backbone.

In one aspect it is disclosed and herein contemplated that the disclosed nucleic acid delivery systems, polymer carriers, and polymer carrier systems form nanoparticles.

The disclosed nucleic acid delivery systems, polymer carriers, and polymer carrier systems disclosed herein are intended to deliver donor corrected nucleic acid to a subject to for example treat CF (for example a donor nucleic acid that corrects for an F508del CFTR mutation). As used herein, nucleic acid can comprise DNA, cDNA, RNA, small interfering RNAs (siRNAs), small hairpin RNAs (shRNAs), antisense molecules, zinc finger nucleases, meganucleases, TAL (TALE) nucleases, triplexes, modified triplexes, wherein the donor-corrected nucleic acid is siRNA. Thus, for example, disclosed herein are nucleic acid delivery systems, comprising donor corrected nucleic acid, wherein the donor corrected nucleic acid is siRNA.

In one aspect it is understood and herein contemplated that the nucleic acid delivery systems, polymer carriers, and polymer carrier systems disclosed herein form and/or deliver nanoparticles. In one aspect, the nanoparticles delivered can be less than 500, 475, 450, 425, 400, 375, 350, 325, 300, 275, 250, 225, 200, 175, 150, 125, 100, 90, 80, 75, 70, 60, 50, 40, or 30 nm in diameter. In one aspect, it is further contemplated that the nucleic acid delivery systems, polymer carriers, and polymer carrier systems disclosed herein can comprise nanoparticles, wherein the nanoparticle formulation comprises a lipid-based nanoparticle.

As disclosed herein, the ratio of moles of the amine group of chitosan to that of the phosphate of the nucleic acid can have profound effect on the stability of the nanocomplex and the ability of the nanocomlex to penetrate mucosal surfaces. The negatively charged mucus have electrostatic interaction with nanocomplexes at high N/P ratios which are positively charged allowing for penetrability. However, as shown herein, while a ratio of one or greater or greater, nanoparticles can permeate mucus, such nanoparticles are not stable. Accordingly, in one, disclosed herein are nucleic acid delivery systems, polymer carriers, and polymer carrier systems, wherein the ratio of moles of the amine groups of chitosan to that of the phosphate of DNA (N/P ratio) is less than 1. For example, the N/P ration can be 0.99, 0.98, 0.95, 0.90, 0.85, 0.8, 0.75, 0.7, 0.65, 0.6, or 0.5. Alternatively, the N/P ration can be between 0.5 and 1, 0.5 and 0.9, or 0.5 and 0.75.

As disclosed herein, the disclosed nucleic acid delivery systems polymer carriers, and polymer carrier systems can be used to deliver nucleic acid which can modify the genome of the human or non-human subject receiving the nucleic acid. Accordingly, disclosed are method of modifying an organism or a non-human organism by manipulation of a target sequence in a genomic locus of interest comprising administering a composition comprising the nucleic acid deliver systems disclosed herein.

In particular, it is contemplated herein that the disclosed nucleic acid systems can be used to deliver gene therapy to a subject and, in particular, the lungs of a subject to correct a genetic disorder, such as, for example cystic fibrosis. Thus, in one aspect, disclosed herein are methods of treating a subject with cystic fibrosis, the method comprising administering the nucleic acid deliver systems disclosed herein.

### Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### EXAMPLES

The following examples are set forth below to illustrate the systems, methods, compositions and results according to the disclosed subject matter. These examples are not intended to be inclusive of all aspects of the subject matter disclosed herein, but rather to illustrate representative systems, methods, compositions and results. These examples are not intended to exclude equivalents and variations of the present invention which are apparent to one skilled in the art.

### Example 1. Chitosan-Derived Nanoparticle-Mediated CRISPR/Cas9 Delivery for the Treatment of Cystic Fibrosis

Disclosed herein are chitosan-based nanoparticles for use in delivering a CRISPR/Cas9 system, useful in a non-viral delivery system for treatment of cystic fibrosis (CF). The development of PEG modified, medium molecular weight chitosan (mPEG-g-Cs) nanoparticles for delivery of CRISPR/Cas9 system to epithelial cells was developed. These particles can be used as carriers for CRISPR/Cas9 system to correct the dF508 CFTR gene in CF patients.

Methods:

### Synthesis of PEG-g-OCs and PEG-g-Cs

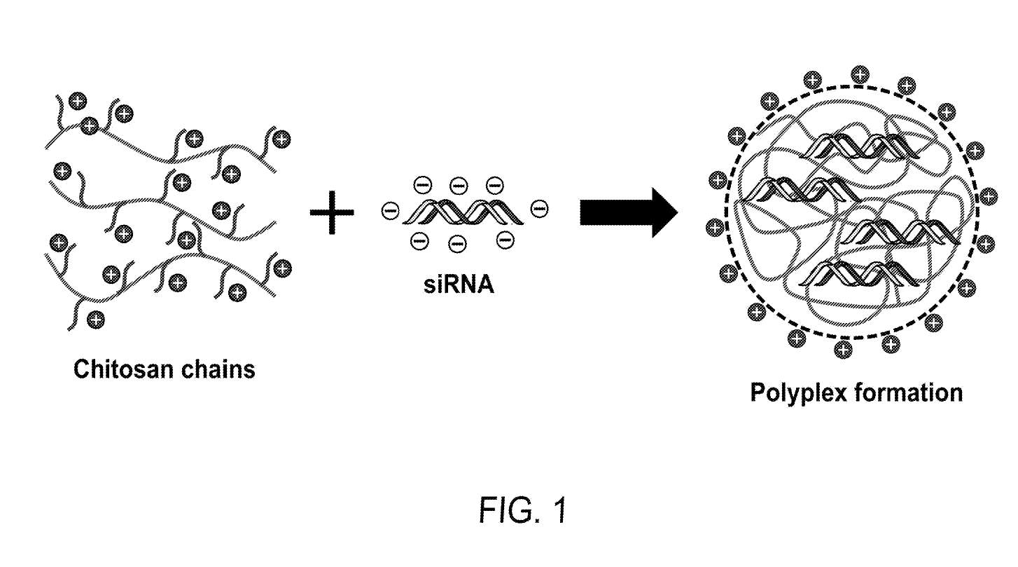

The synthesis of the chitosan derivatives PEG-g-OCs (oligo-saccharide) and PEG-g-Cs (chitosan medium molecular weight) was carried out. Initially, the NH₂ groups of chitosan (10 g) were protected by treating chitosan with phthalic anhydride (27.6 g) in 200 ml of DMF at 120-130° C. for approximately 8 h in a nitrogen atmosphere. After the reaction, the product was poured into ice water to obtain a precipitate, which was filtered and washed with methanol. The N-phthaloyl oligochitosan and N-phthaloyl chitosan (NPHOCs and NPHCs) products were finally lyophilized to obtain a powder sample. FIG. 1 shows an illustration of complex formation using the systems PEG-g-OCs and PEG-g-Cs and siRNA.

mPEG (20 g) was converted into mPEG-COOH when reacted with succinic anhydride (30 g) in 100 ml of pyridine. The mixture was stirred at 50-60 □ for 50 h in a nitrogen atmosphere. Aliquots of succinic anhydride (10 g each) were added as a dry powder at 1-hr intervals. Following the last addition, the reaction mixture was stirred for 2 hr at 50-60 □. The mPEG-COOH was precipitated in diethyl ether and filtered. Finally, the product was dialyzed against distilled water to remove unreacted succinic anhydride and remaining diethyl ether.

The compounds NPHOCs and NPHCs were reacted with mPEG-COOH, EDC. HCl, HOBt and DMAP in at 50-60 □ for 24 h. The products mPEG-g-NPHOCs and mPEG-g-NPHCs were purified by dialysis against water and washed with ethanol to remove impurities. The final solution was then lyophilized to obtain dry powder. Finally, the N-phthalimido group was removed by treating mPEG-g-NPHOCs or mPEG-g-NPHCs (4 g) with hydrazine monohydrate (15 ml) in DMF (15 ml) at 100° C. for 2 h. The products mPEG-g-OCs and mPEG-g-Cs were purified by dialysis against water and lyophilized.

### Preparation of PEG-g-OCs/DNA and PEG-g-Cs/DNA Complexes

PEG-g-OCs/DNA and PEG-g-Cs/DNA complexes having a DNA concentration of 50 μg/ml with different N/P ratios were prepared by the self-assembly and adapted to a final volume of 500 μl. Briefly, the DNA stock solution was diluted in sodium acetate/acetic acid buffer (pH 5.5). PEG-g-OCs and PEG-g-Cs nanoparticles was suspended in the same buffer to form a solution of 50 mg/ml and 100 mg/ml respectively. A series of complexes at various N/P ratios were prepared by mixing chitosan solutions with a DNA solution, vortexing for 3 s and incubated for 30 min at room temperature before using.

### Analysis of Particle Size and Zeta Potential

The hydrodynamic size of PEG-g-OCs/DNA and PEG-g-Cs/DNA complexes was measured by Zetasizer Nano ZS analyzer (Malvern Instrument, UK). Once prepared, the complexes were further diluted in sodium acetate/acetic acid buffer solution (pH 5.5) and analyzed. To determination of the zeta potential the complexes were diluted in sodium acetate/acetic acid buffer pH 5.5 (to maintain the stability of the complexes), and transferred to capillary cuvettes for the detection of particle size and zeta potential.

Results:

### Synthesis and Characterization of PEG-g-OCs and PEG-g-Cs

The synthesis of the PEG-g-OCs and PEG-g-Cs systems was carried out using oligochitosan (OCs, 15 kDa) and chitosan medium molecular weight (Cs, 190-310 kDa) as well as PEG (5 kDa). As described in Scheme 1, the N-phthaloyl oligochitosan (NPHOCs) and N-phthaloyl chitosan (NPHCs) were successfully grafted with mPEG-COOH to obtain the products PEG-g-NPHOCs and PEG-g-NPHCs, respectively.

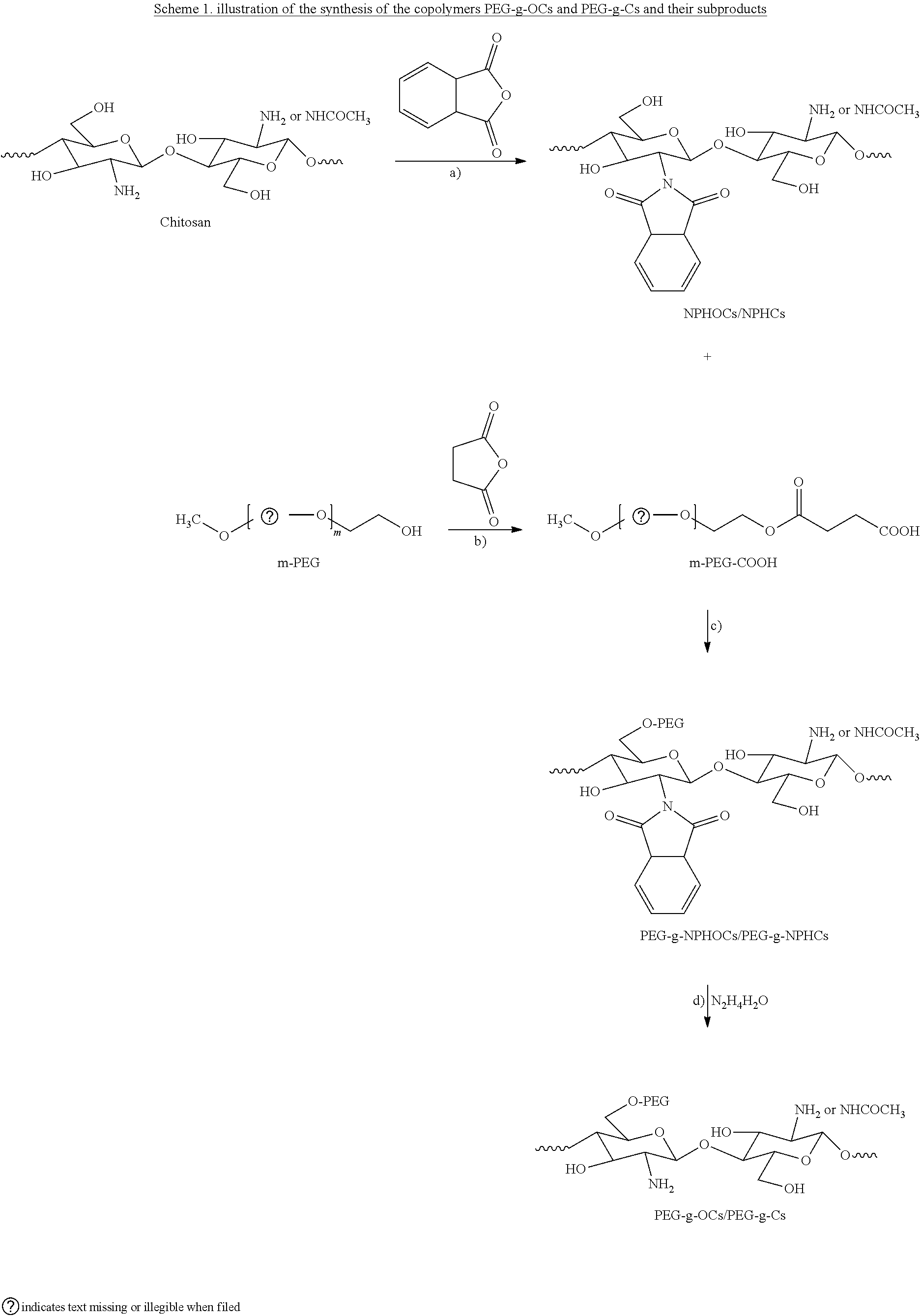

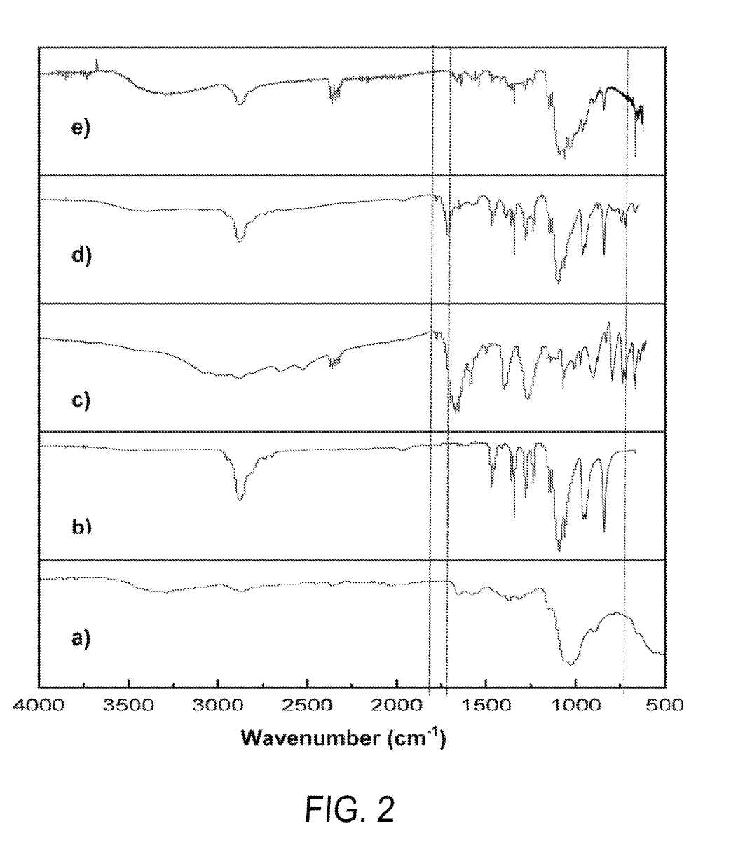

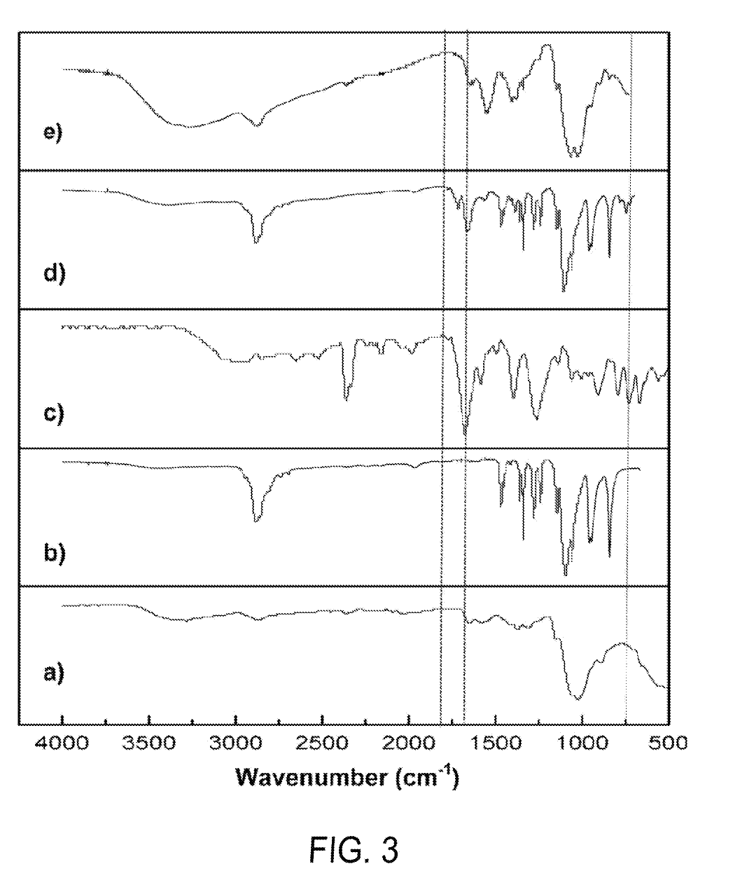

The products were confirmed by FT-IR by the presence of absorption bands at 1775 (imide), 1694 (imide) and 720 (aromatic ring) cm-1 corresponding to the N-phthaloylation of oligochitosan (NPHOCs)(FIG. 2c), and 1775, 1698, and 732 cm-1 corresponding to the N-phthaloylation of chitosan (FIG. 3c). The FT-IR spectra of the PEG-g-NPHOCs and PEG-g-NPHCs (see FIGS. 2d and 3d) showed the absorption bands corresponding to the phthaloyl groups in addition to the PEG bands at 2879 (stretching C—H), 1100 cm-1 (stretching C—O), and 1059 (stretching C—O—C). The final copolymers, PEG-g-OCs and PEG-g-Cs, were obtained by the deprotection of the NH2 groups of chitosan using hydrazine monohydrate. The synthesis of both copolymers was confirmed by FT-IR, where the spectra showed the absence of the bands corresponding to the phthaloyl groups (FIGS. 2e and 3e).

### Preparation and Characterization of mPEG-g-Cs/DNA Complexes

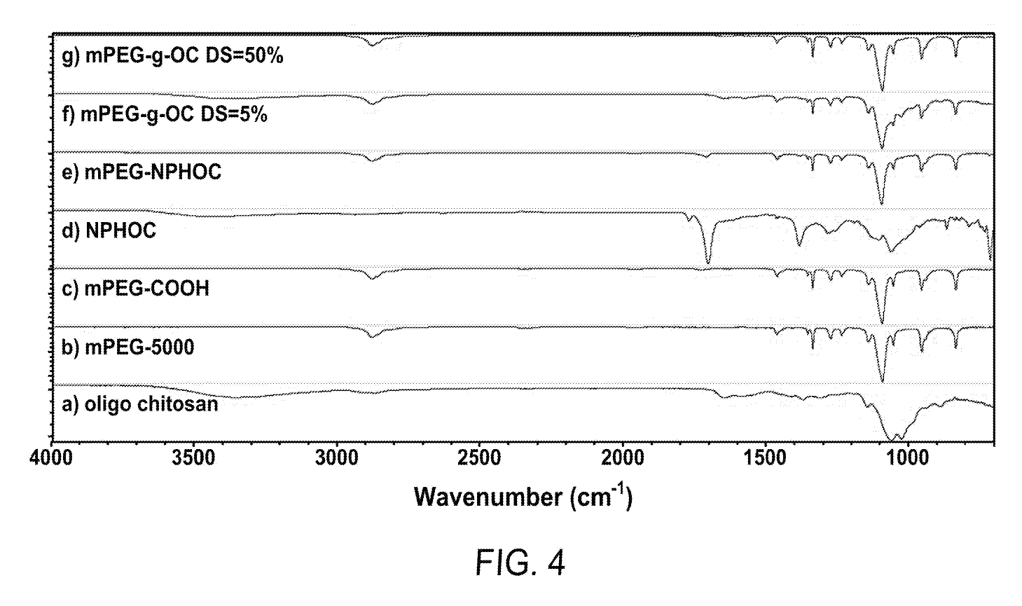

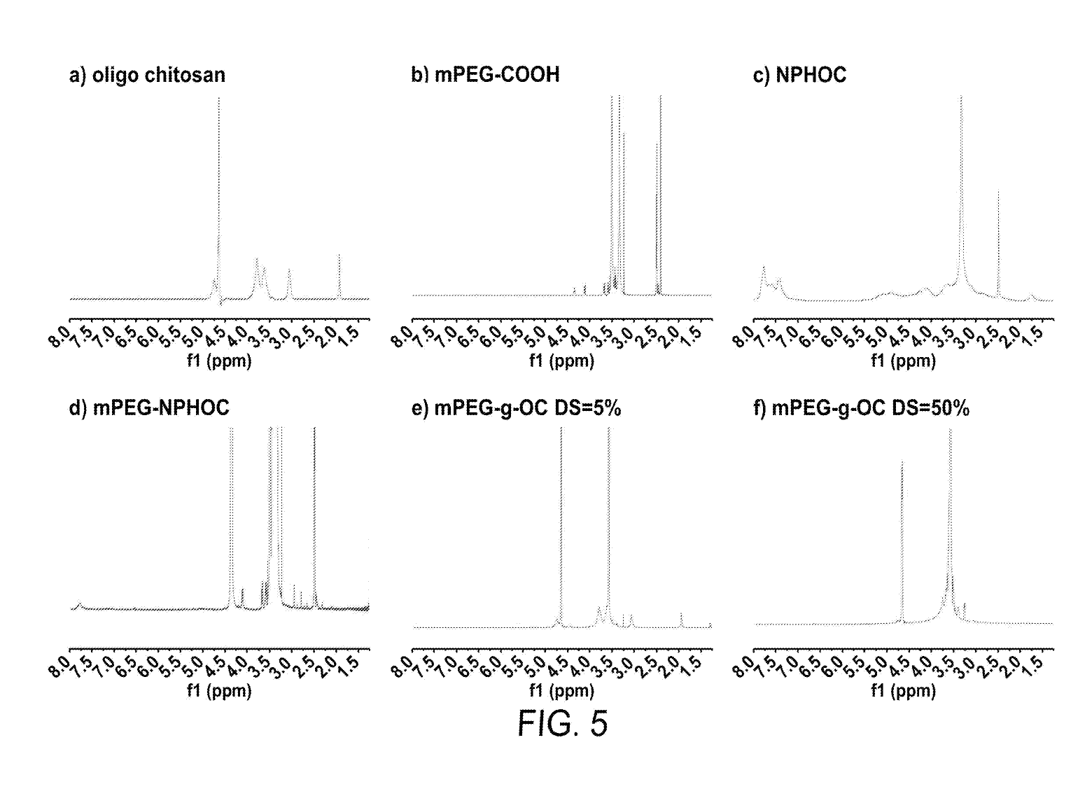

mPEG-g-Cs/DNA (pSpCas9-2A-GFP) complexes were prepared by adding DNA solution drop-wise into mPEG-g-Cs solution to achieve different charge ratios. The characteristics of each compound and zeta potential were measured. Hydrodynamic size of mPEG-g-Cs/DNA complexes were measured by dynamic light scattering. Gel retardation assays were used to determine loading efficiencies. Synthesis of PEG-g-OC with different degree of PEG substitution (DS) by using a novel method described above. The products were detected by FTIR (FIG. 4) and H¹ NMR (FIG. 5).

Gel Retardation Assays

During the experiment, the N/P ratios (ratio of moles of the amine groups of chitosan to that of the phosphate of DNA) 2:1, 5:1, 10:1, 20:1, and 30:1 were evaluated. The retention or immobilization of the DNA in the well during the agarose gel electrophoresis was used as an indication complex formation.

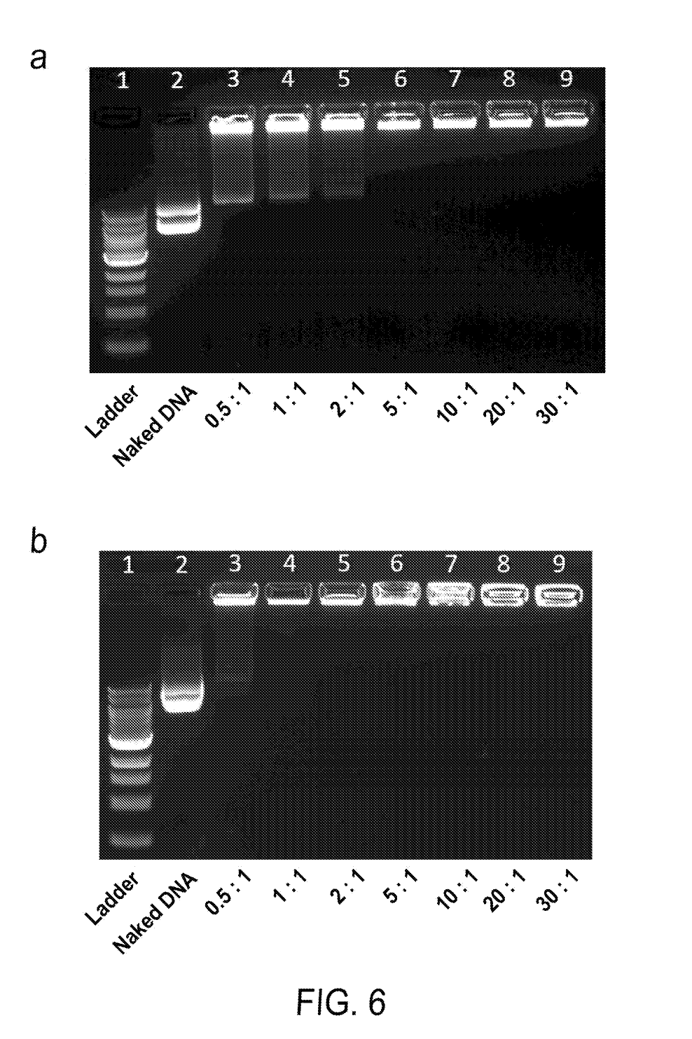

FIG. 6 shows that both mPEG-g-OCs and mPEG-g-Cs can form complexes with DNA but only partial retention and condensation of DNA was seen at N/P ratio=0.5, 1 and 2 in mPEG-g-OCs and at N/P ratio=0.5 at mPEG-g-Cs. The capacity of the system to efficiently complex the DNA molecule was observed at the ratios=5, 10, 20 and 30 in mPEG-g-OCs and at N/P ratios=1, 2, 5, 10, 20 and 30 in mPEG-g-Cs, where complete retardation of the DNA migration in the gel was achieved. At these ratios, the DNA was tightly bound and condensed into the PEG-g-Cs copolymer chains, resulting in complexes formation (cationic polymer/DNA complexes) formation. In contrast, no retention was seen when using naked DNA (FIGS. 6a and 6b, lane 2). These results demonstrated that mPEG-g-Cs have a higher DNA encapsulation efficiency than mPEG-g-OCs.

Confirmation of both PEG-g-OCs and PEG-g-Cs of polyplex formation and loading efficiency of siRNA showed complete retardation of the siRNA migration at 1:80, 1:120, 1:150, and 1:180. The 1:120 (w/w) ratio was selected as the optimal ratio to prepare polyplexes for further assays due to loading efficiency of both polyplexes.

The polyplex stability at 1:120 siRNA:polymer ratio was determined using photon correlation spectroscopy (mean hydrodynamic size) and zeta potentials. The assays showed that particle size of nanoparticles contributes to the cellular interaction and uptake. Smaller size of PEG-g-OCs was due to higher degree of deacetylation (95%) of oligochitosan. This smaller size facilitated a stronger interaction with siRNA, making the complex smaller. Thus, the deacetylation and molecular weight of chitosan can play a key role in controlling the size required to facilitate cellular uptake. Also observed was that the complex is preferably positively charged in order to internalize into the cells. Positive values measured demonstrate formation of stable complexes. The higher zeta potential for PEG-g-OCs was due to higher degree of deacetylation.

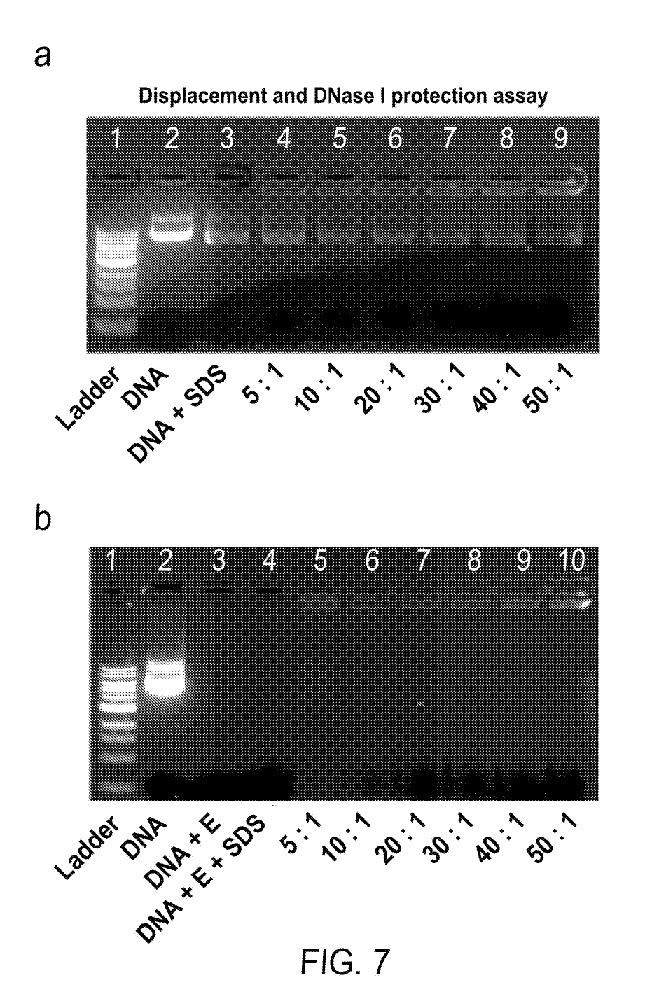

To show that DNA successfully released from the complexes, a displacement assay was performed by using sodium dodecyl sulfate (SDS, final concentration was 2.5%, incubated at room temperature for 24 h)(FIG. 7A). This indicates that the interaction between the copolymer and the DNA can be strong enough to form stable complexes but sufficiently tenuous to allow detachment of DNA from the complex to exert its biological activity. A trend of decrease in the released DNA as N/P ratio increased was also observed.

Additionally, a protection capacity against DNase I enzymatic digestion was conducted (FIG. 7B). E (enzyme) stands for DNase I (0.3 U at 37 degree for 15 min and then 75 degree to inactivate). Lane 1 represents ladder and lane 2 represents naked DNA. Lane 3 and Lane 4 represent naked DNA (640 ng) and naked DNA treated with DNase I respectively. Lanes 5-10 represent N/P ratios=5, 10, 20, 30, 40 and 50 respectively. The absence of signal in lane 3 and 4 indicates that the enzyme worked properly. In lanes 5-10, the signal near wells represent DNA remained intact in complexes at different ratio, which indicates that the complex have the ability to protect DNA from DNase I digestion.

Measurements of Size and Zeta Potential

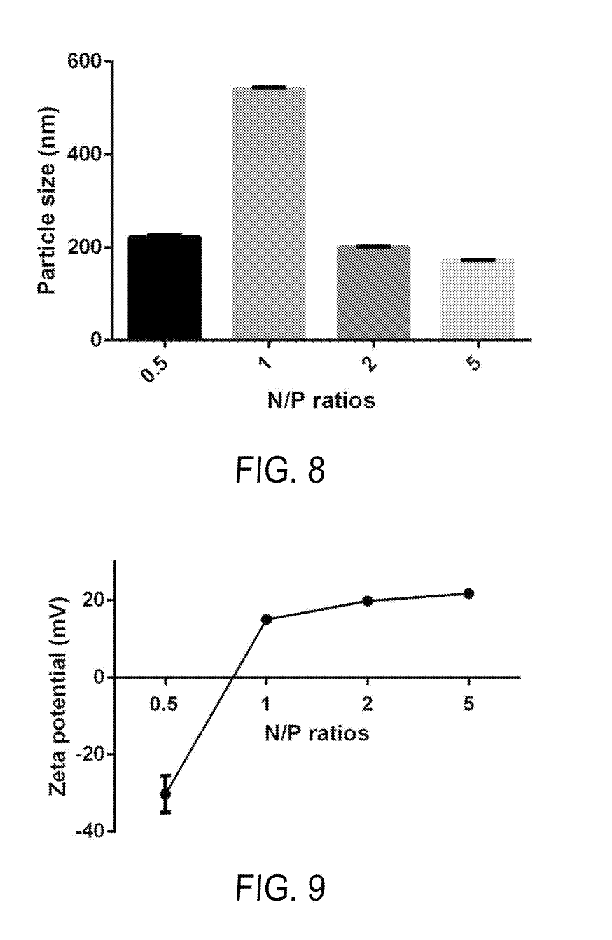

The average size and zeta potential of complexes are shown in FIG. 8 and FIG. 9 respectively. In general, the particle size decreased as N/P ratio increased (from 222.7±4.8 nm at N/P=0.5 to 171.6±2.4 nm at N/P=5), indicating the charge ratio affected the size of complex. It was worth to note that aggregate was observed at N/P ratio=1 since the nanocomplex was neutrally charged at this N/P ratio. The zeta potential was not substantially affected by the N/P ratio. Zeta potential values oscillated from 15.1±0.1 mV at N/P=1 to 21.8±1.1 mV at N/P=5. The most obvious change in zeta potential value was observed when N/P ratio raised from 0.5 to 1.

Mucus Penetration Assay

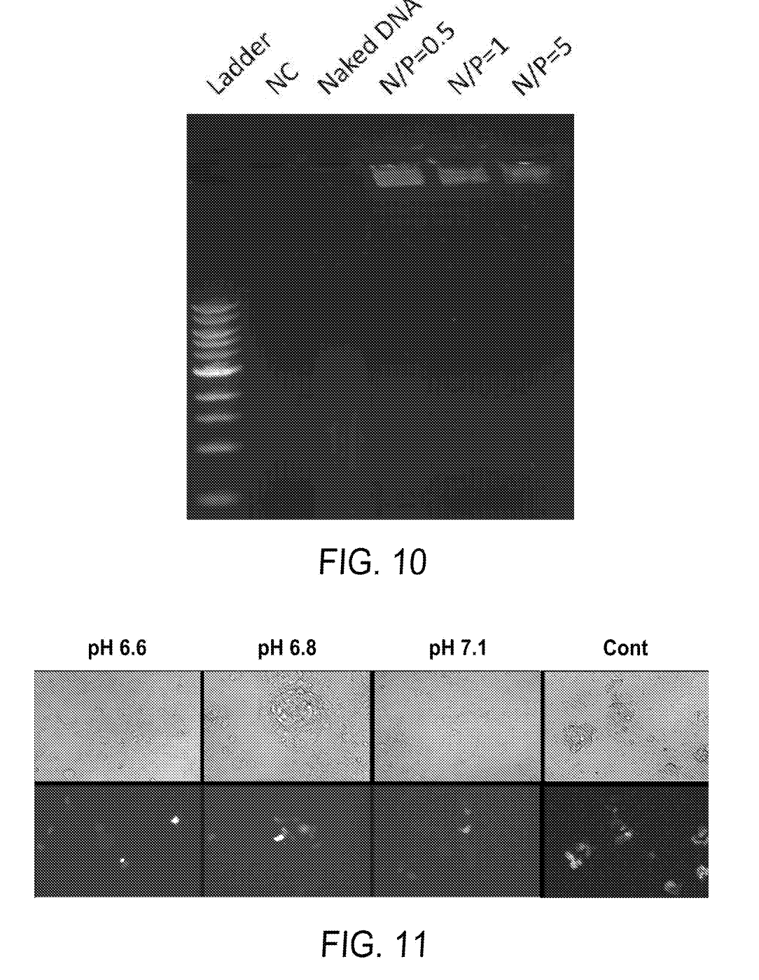

Mucus model: final concentration of mucin was 1.6 mg/ml. After addition of 20 μl of 8 mg/ml mucin solution on the top of the filter membrane in a transwell insert and 600 μl of distilled water in the reservoir, 80 μl of sodium acetate buffer (pH 5.5), naked DNA, nanocomplexes at different N/P ratios (DNA concentration was 50 ng/μl) were added to the top of the mucus in each transwell. After incubation at 37° C. for 6 h, 100 μl of sample was collected from the reservoir and loaded into agarose gel. In FIG. 10, bands in lane 4, 5 and 6 showed that the nanocomplexes at N/P ratios=0.5, 1 and 5 have the ability to penetrate mucus and the penetration ability decreased as N/P ratio increased. This is partially due to that the negatively charged mucus have electrostatic interaction with nanocomplexes at high N/P ratios which are positively charged. In conclusion, PEG-g-Cs/DNA nanocomplex have the ability to penetrate mucus with the optimal penetration at N/P ratio range from N/P ratio 0.5-1. These results show an unexpected and narrow range of operability for at least one example of the delivery system. Essentially for peg-chitosan the ratio (N to P, or polymer to nucleic acid) is best below 1. At a ratio of 1 the Nanoparticles permeate mucus, but it can be seen their sizes indicate instability. By contrast, np ratio less than 1 had better mucus permeation and better sizes indicative of stability.

Transfection Assay 98. During this study, the HEK293 cell line and A549 cell line were used as in vitro models since they are commonly used to investigate drug metabolism and cell transfection efficiency. HEK293 cells and A549 cells were maintained in Dulbecco's modified Eagle medium (DMEM) and Kaighn's Modification of Ham's F-12 Medium (F-12K) respectively, which were supplemented with 10% fetal bovine serum (FBS), 100 U penicillin/0.1 mg/mL streptomycin at 37° C. in a humidified 95% air and 5% CO2 atmosphere.

The transfection efficiency of the DNA plasmid pSpCas9(BB)-2A-GFP was evaluated in HEK293 and A549 cells. In brief, 7500 HEK293 and A549 cells were seeded in 96 well-plates and incubated overnight to allow adherence in DMEM and F12K medium. After incubation, the medium was removed and Opti-MEM serum-free media was added to the cells. DNA plasmid liposomes were prepared as follows: for each transfected well 200 ng of DNA and 0.2 μl of P3000 were diluted in 10 μl Opti-MEM serum reduced media, and 0.3 l of lipofectamine 3000 was diluted in 10 μl of Opti-MEM serum reduced media. Both solutions were mixed together and incubated for 15 min at room temperature. The final solution was added to the cells. For complexes, 10 μl of complexes (300 ng of DNA plasmid) at different N/P ratios were add to cells cultured in media with different pH (6.6, 6.8 and 7.1). After incubation for 48 h, the transfection efficiency was evaluated by fluorescence microscopy and flow cytometry.

The results showed that complexes at N/P ratios=10, 20 and 30 successfully delivered pSpCas9-2A-GFP to cells cultured in media with pH 6.6, 6.8 and 7.1 (FIG. 11). Herein was demonstrated that CRISPR/Cas9 gene editing system can be delivered by the chitosan-derived nanoparticles to epithelial cells. These promising results indicate that mPEG-g-Cs/DNA systems represent a safe alternative to viral and toxic systems for CRISPR/Cas9 cystic fibrosis treatment.

Cytotoxicity

To measure cytotoxicity a cytotoxicity assay of PEG-g-OCs and PEG-g-Cs complexes were performed in A549 cells. Importantly, no significant difference was observed between control cells and PEG-g-OCs at low concentration and PEG-g-Cs at high and low concentrations. The significant difference observed in the PEG-g-OCs at high concentration was still relatively low compared to other frequently used agents.

Cellular Internalization of FAM-Labeled siRNA

Cellular internalization of FAM-labeled siRNA by PEG-g-OCs and PEG-g-Cs complexes at 1:120 ratio was performed in A549 cells after 4 h of incubation and measured by flow cytometry. The results showed that cells did not internalize the naked siRNA (only 2% fluorescence). By contrast, PEG-g-OCs were highly internalized (99% internalization). Higher than the positive control. However, PEG-g-Cs failed to increase internalization (8%). Lower than the positive control (>80%). Possibly attributed to the weak interactions with the cellular membrane and/or polyplex charge neutralization due to low zeta potential.

### CONCLUSIONS

Three non-viral polyplex systems, PEG-g-OCs+siRNA, PEG-g-Cs+siRNA and PEG-g-Cs+pSpCas9-2A-GFP, were synthesized through electrostatic interactions. These polyplex systems are biodegradable, present low cytotoxicity, and are resistant to enzymatic degradation up to 24 hours. The PEG-g-OCs+siRNA polyplex system significantly increased cellular uptake of siRNA resulting in a significant increase in silencing activity. Additionally, the PEG-g-Cs+pSpCas9-2A-GFP polyplex successfully delivered CRISPR/Cas9 gene editing system into cells.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

### REFERENCES

Atassi, M. Z. and T. Manshouri, Synthesis of tolerogenic monomethoxypolyethylene glycol and polyvinyl alcohol conjugates of peptides. J Protein Chem, 1991, 10(6): p. 623-7.
Camarasa M V, Galvez V M. Robust method for TALEN-edited correction of pF508del in patient-specific induced pluripotent stem cells. Stem cell research & therapy. 2016; 7(1):26. doi: 10.1186/s13287-016-0275-6.
Cantin A M, Hartl D, Konstan M W, Chmiel J E Inflammation in cystic fibrosis lung disease: Pathogenesis and therapy. Journal of cystic fibrosis: official journal of the European Cystic Fibrosis Society. 2015; 14(4):419-30. doi: 10.1016/j.jcf.2015.03.003.
Crane A M, Kramer P, Bui J H, Chung W J, Li X S, Gonzalez-Garay M L, et al. Targeted correction and restored function of the CFTR gene in cystic fibrosis induced pluripotent stem cells. Stem cell reports. 2015; 4(4):569-77. doi: 10.1016/j.stemcr.2015.02.005.
Firth A L, Menon T, Parker G S, Qualls S J, Lewis B M, Ke E, et al. Functional Gene Correction for Cystic Fibrosis in Lung Epithelial Cells Generated from Patient iPSCs. Cell reports. 2015; 12(9):1385-90. doi: 10.1016/j.celrep.2015.07.062.
Guzman-Villanueva D, El-Sherbiny I M, Vlassov A V, Herrera-Ruiz D, Smyth H D. Enhanced cellular uptake and gene silencing activity of siRNA molecules mediated by chitosan-derivative nanocomplexes. International journal of pharmaceutics. 2014; 473(1-2): 579-90. doi: 10.1016/j.ijpharm.2014.07.026.
Hutt D M, Herman D, Rodrigues A P, Noel S, Pilewski J M, Matteson J, et al. Reduced histone deacetylase 7 activity restores function to misfolded CFTR in cystic fibrosis. Nature chemical biology. 2010; 6(1):25-33. doi: 10.1038/nchembio.275.
Kim T-H, Jiang H-L, Jere D, Park I-K, Cho M-H, Nah J-W, et al. Chemical modification of chitosan as a gene carrier in vitro and in vivo. Progress in Polymer Science. 2007; 32(7):726-53. doi: 10.1016/j.progpolymsci.2007.05.001.
Kotha K, Clancy J P. Ivacaftor treatment of cystic fibrosis patients with the G551D mutation: a review of the evidence. Therapeutic advances in respiratory disease. 2013; 7(5):288-96. doi: 10.1177/1753465813502115.
Lee C M, Flynn R, Hollywood J A, Scallan M F, Harrison P T. Correction of the DeltaF508 Mutation in the Cystic Fibrosis Transmembrane Conductance Regulator Gene by Zinc-Finger Nuclease Homology-Directed Repair. BioResearch open access. 2012; 1(3):99-108. doi: 10.1089/biores.2012.0218.
Mao S, Sun W, Kissel T. Chitosan-based formulations for delivery of DNA and siRNA. Advanced drug delivery reviews. 2010; 62(1): 12-27. doi: 10.1016/j.addr.2009.08.004.
McNeer N A, Anandalingam K, Fields R J, Caputo C, Kopic S, Gupta A, et al. Nanoparticles that deliver triplex-forming peptide nucleic acid molecules correct F508del CFTR in airway epithelium. Nature communications. 2015; 6:6952. doi: 10.1038/ncomms7952.
O'Sullivan B P, Freedman S D. Cystic fibrosis. Lancet (London, England). 2009; 373(9678): 1891-904. doi: 10.1016/s0140-6736(09)60327-5.
Schwank G, Koo B K, Sasselli V, Dekkers J F, Heo I, Demircan T, et al. Functional repair of CFTR by CRISPR/Cas9 in intestinal stem cell organoids of cystic fibrosis patients. Cell stem cell. 2013; 13(6):653-8. doi: 10.1016/j.stem.2013.11.002.
Van Goor F, Hadida S, Grootenhuis P D, Burton B, Cao D, Neuberger T, et al. Rescue of C F airway epithelial cell function in vitro by a CFTR potentiator, VX-770. Proceedings of the National Academy of Sciences of the United States of America. 2009; 106(44):18825-30. doi: 10.1073/pnas.0904709106.
Van Goor F, Hadida S, Grootenhuis P D, Burton B, Stack J H, Straley K S, et al. Correction of the F508del-CFTR protein processing defect in vitro by the investigational drug VX-809. Proceedings of the National Academy of Sciences of the United States of America. 2011; 108(46):18843-8. doi: 10.1073/pnas.1105787108.

What is claimed is:

1. A polymer carrier comprising polyethylene glycol (PEG) chemically bound to a chitosan or oligo-chitosan backbone

2. A nucleic acid delivery system comprising:
a. a CRISPR-Cas9 polynucleotide sequence, wherein the polynucleotide sequence comprises a guide sequence capable of hybridizing to a target sequence in a eukaryotic cell;
b. donor-corrected nucleic acid; and
c. a polymeric carrier system, wherein the polymeric carrier comprises polyethylene glycol (PEG) bound to a chitosan or oligo-chitosan backbone.

3. The nucleic acid delivery system of claim 2, wherein the delivery system forms nanoparticles.

4. The nucleic acid delivery system of claim 2, wherein the donor-corrected nucleic acid is siRNA.

5. The nucleic acid delivery system of claim 2, wherein the donor-corrected nucleic acid corrects an F508del CFTR mutation.

6. The nucleic acid delivery system of claim 2, wherein the delivery particles are less than 500 nm in diameter.

7. The nucleic acid delivery system of claim 2, wherein the delivery particles are less than 250 nm in diameter.

8. The nucleic acid delivery system of claim 2, wherein the delivery particles are less than 100 nm in diameter.

9. The nucleic acid delivery system of claim 2, wherein the nanoparticle formulation comprises a lipid-based nanoparticle.

10. The nucleic acid delivery system of claim 2, wherein the ratio of moles of the amine groups of chitosan to that of the phosphate of DNA (N/P ratio) is less than 1.

11. A method of modifying an organism or a non-human organism by manipulation of a target sequence in a genomic locus of interest comprising administering a composition comprising the nucleic acid delivery system of claim 2.

12. A method of treating a subject with cystic fibrosis, the method comprising administering the nucleic acid delivery system of claim 4.
